(19) United States
(12) Patent Application Publication
(10) Pub. No.: US 2003/0192534 A1
(43) Pub. Date: Oct. 16, 2003

(54) Medicament dispenser

(76) Inventors: Stefan Klump, Lichtenfels (DE); Klaus Van Der Linden, Unterlangenstadt (DE)

Correspondence Address: LERNER AND GREENBERG, P.A., POST OFFICE BOX 2480, HOLLYWOOD, FL, 33022-2480, US

(21) Appl. No.: 10/410,384

(22) Filed: Apr. 9, 2003

Related U.S. Application Data
(63) Continuation of application No. PCT/DE01/03689, filed on Sep. 26, 2001.

(30) Foreign Application Priority Data
Oct. 9, 2000 (DE) 100 49 820.5

(51) Int. Cl. A61M 11/00

(52) U.S. Cl. 128/200.23

(57) ABSTRACT

The invention relates to a medicament dispenser including a dosing device and an indicator device. The indicator device sums up a use-related value that is associated with every actuation of the dosing device, calculates a value indicating the total number of uses, and displays the value.

### CROSS-REFERENCE TO RELATED APPLICATION

This application is a continuation of copending International Application No. PCT/DE01/03689, filed Sep. 26, 2001, which designated the United States and was not published in English.

### BACKGROUND OF THE INVENTION

### FIELD OF THE INVENTION

The invention relates to a medicinal medicament dispenser.

In medicine, medicament dispensers are often used which the patient actuates in order to take a required medicament.

Such medicament dispensers can, for example, be an inhaler device or an injection device, for example, for insulin or painkillers.

An important object of such a medicament dispenser is to ensure that, when the dispenser is actuated by the patient, the medicament is administered in an exact dose, so as to guarantee the efficacy of the medicament and to rule out any risks. From Published European Patent Application EP 0 642 802 B1 and Published European Patent Application EP 0 814 860 B1, an inhaler device is known which ensures the delivery of liquid medication and its optimum atomization, in order to guarantee exactly the desired dose when the inhaler device is actuated. U.S. Pat. No. 2,605,765 discloses an automatic medical syringe from which a medicament is delivered continuously and the amount delivered or the duration of delivery is indicated. U.S. Pat. No. 5,497,764 discloses a medicament dispenser in which, inter alia, the number of the individual actuation of the medicament dispenser is electronically detected and the actuations remaining as a function of the residual amount of the medicament are indicated.

Such devices do not guarantee that, upon each actuation during the lifetime of the device, a dose of medicament will be dispensed which lies within a permissible tolerance.

### SUMMARY OF THE INVENTION

It is accordingly an object of the invention to provide a medicament dispenser, which overcomes the above-mentioned disadvantages of the prior art apparatus of this general type.

In particular, it is an object of the invention to provide a medicament dispenser that, during its lifetime, guarantees dosing of the medicament within permissible tolerance limits.

With the foregoing and other objects in view there is provided, in accordance with the invention, a medicament dispenser including: a dosing device, and an indicator device for forming and displaying a total usage value obtained by totaling at least one usage value assigned to each actuation of the dosing device. The indicator device is designed for indicating when the total usage value exceeds a minimum total usage value. The minimum total usage value is derived from an operating period during which there is a high degree of probability that the medicament dispenser operates free from error.

In accordance with an added feature of the invention, the minimum total usage value has been determined in advance by performing a test series.

In accordance with an additional feature of the invention, a medicament container is connected to the dosing device and to the indicator device.

In accordance with another feature of the invention, the dosing device includes at least one motor connected to the indicator device.

In accordance with a further feature of the invention, a gear mechanism connects the indicator device to the motor of the dosing device.

In accordance with a further added feature of the invention, a medicament container is connected to the dosing device and to the indicator device. The indicator device is designed as a counting device that forms the total usage value by determining a total amount of medicament removed from the medicament container.

In accordance with another added feature of the invention, the indicator device is designed as a counting device that forms the total usage value by determining a total period of use.

In accordance with another additional feature of the invention, the indicator device includes an indicator disk for displaying a current value of the total usage value.

In accordance with yet an added feature of the invention, the indicator disk is divided into at least two areas each assigned to a value or a value range of the total usage value.

The object of the invention is achieved by a medicament dispenser with a dosing device and an indicator device which, by totaling at least one usage value which is assigned to each actuation of the dosing device, forms and displays a total usage value. The indicator device is designed to indicate when a minimum total usage value is exceeded. The minimum total usage value is derived from an operating period of the device during which there is a high degree of probability that the medicament dispenser operates free from error.

The advantage of this medicament dispenser lies in particular in the fact that the total usage value formed permits a conclusion to be drawn as to whether the medicament dispenser is still functioning in the desired way and in particular as to whether it still guarantees the correct dosage of the medicament.

The medicament itself is usually provided with a date that indicates the time until which the efficacy of the medicament is guaranteed.

The medicament dispenser now additionally affords safety in terms of whether the device itself, which is intended to deliver the medicament to the patient, is functioning reliably.

A minimum total usage value can be derived, for example, from the design and construction of the medicament dispenser and can, for example, be a total dose of the medicament that can be administered in individual doses with high probability during the lifetime of the medicament dispenser without incorrect functioning of the medicament dispenser occurring.

If the total usage value exceeds a minimum total usage value, this is displayed by the indicator device. The patient is thus given a valuable indicator that in some circumstances the correct functioning of his medicament dispenser may no longer be guaranteed. He is thus able, in good time, to have his medicament dispenser inspected in order to avoid an impending deterioration or even loss of its function.

The minimum total usage value is advantageously determined in advance by performing a test series.

In an advantageous embodiment, the dosing device and the indicator device are connected to a medicament container.

In this embodiment of the invention, the indicator device totals a usage value which is assigned to each actuation of the dosing device, and as a result of the connection to the medicament container, additionally obtains information on whether the cumulative usage value corresponds to the conditions in the medicament container, for example, the current filling level of the medicament container. Thus, for example, it is possible to determine whether the medicament container has an undesirable leak or whether the cumulative individual usage values, which each correspond to an individual dose of the medicament, have led to a total usage value which, in the case of the incorrect functioning of the medicament dispenser, may be too high or too low and thus no longer corresponds to the residual amount of medicament remaining in the medicament container.

In a further advantageous embodiment of the invention, the dosing device includes at least one motor and the indicator device is connected to this motor.

As, for example, in the prior art already cited, a medicament dispenser can be provided with a motor which, upon each actuation of the dosing device, guarantees the accuracy of the administered dose of medicament. The time during which the motor runs upon an actuation of the dosing device is a measure of the dose of the medicament administered on each actuation. In this embodiment of the invention, the indicator device thus totals a usage value that corresponds to the respective running time of the motor upon an actuation of the dosing device and is thus a measure of the total dose of the medicament that has already been administered.

The indicator device is advantageously connected to the motor of the dosing device via a gear mechanism.

The gear mechanism ensures that the movements of the motor shaft are optimally converted for recording by the indicator device. Depending on the transmission ratio of the gear mechanism, the recording accuracy of the indicator device can, for example, be influenced in this way. In addition, the transmission ratio of the gear mechanism plays a role in the calibration of the display of the total usage value by the indicator device.

Test series are carried out on identical medicament dispensers to determine the respective running time of the devices during which the patient with great certainty does not obtain a dose of medicament deviating from a desired normal value. The running times determined for the individual tested medicament dispensers are then processed using known statistical methods. One result can, for example, be a statistically determined total minimum usage value corresponding to a total medicament dose which with a high degree of probability can be dispensed from a medicament dispenser of the type tested. A statistically determined minimum total usage value can also be, for example, the total running time of the motor of the dosing device during which the motor with a high degree of probability operates free from error.

In a further advantageous embodiment of the invention, the indicator device is designed as a counting device that, during the operation of the medicament dispenser, determines, as a usage value, the total amount of medicament removed and/or the total period of use.

Both of the stated forms of the usage value provide the patient with a valuable indicator of whether his medication is still guaranteed to the desired degree. The total amount of medicament removed is in this case a measure of the total medicament dose administered, and the total usage period is a measure of how long the medicament dispenser has been actively used.

In a further advantageous embodiment of the invention, the indicator device includes an indicator disk from which the current total usage value can be read off.

In this embodiment of the invention, the patient can easily read off the current total usage value.

The indicator disk is advantageously divided into at least two areas that are each assigned to a value or a value range of the total usage value.

In this way, the information to be read off by the patient can be compressed to such an extent that only the information of greatest importance to the patient can be read off. For example, the indicator disk is divided into a green area and a red area, which areas correspond, respectively, to “Medicament dispenser is working correctly” and “Medicament dispenser may no longer be working correctly”. If the patient now reads off the current total usage value “green” from the indicator disk, he knows that his medicament dispenser is, with a high degree of probability, still functioning correctly, whereas if he reads off the total usage value “red” from the indicator disk, he knows that it is necessary to have his medicament dispenser inspected. These two important forms of information are generally sufficient for the patient; it is in most cases unnecessary to represent the total usage value in greater detail.

Other features which are considered as characteristic for the invention are set forth in the appended claims.

Although the invention is illustrated and described herein as embodied in a medicament dispenser, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 is a longitudinal cross sectional view taken through a medicament dispenser designed as an inhaler device; and

FIG. 2 is a longitudinal cross sectional view taken through a medicament dispenser designed as an injection device.

### DESCRIPTION OF THE PREFERRED EMBODIMENTS

In the figures, identical parts are provided with the same reference numbers.

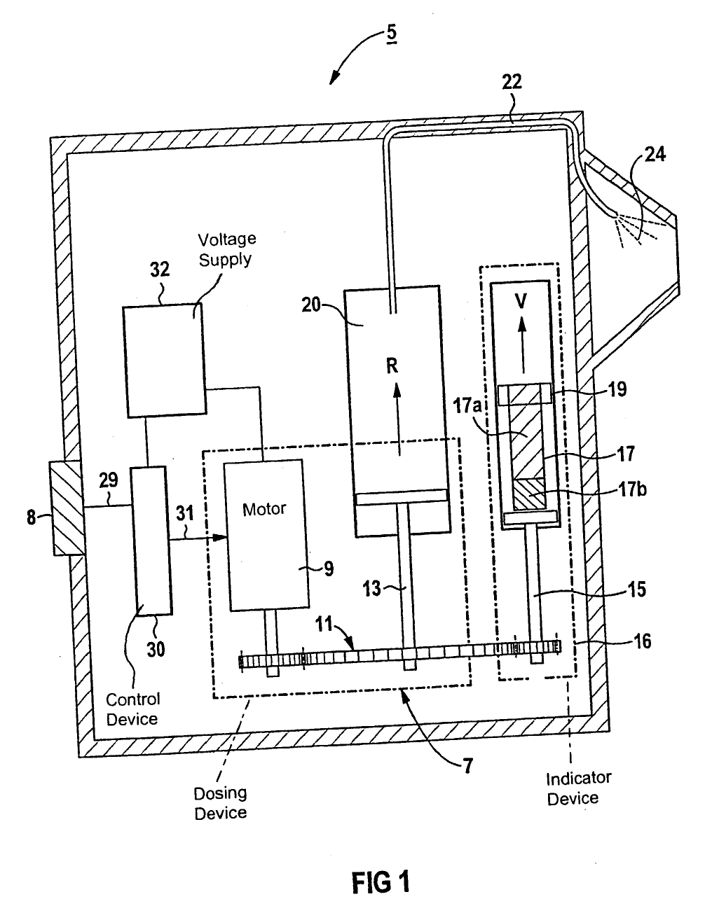

Referring now to the figures of the drawing in detail and first, particularly, to FIG. 1 thereof, there is shown a medicament dispenser 5 having a dosing device 7 including a motor 9, a gear mechanism 11, and a dosing piston 13.

The patient triggers the delivery of a dose of the medicament by actuating the actuating button 8, and this action is conveyed via the sensor line 29 to the control device 30. The control device 30 which is connected to the voltage supply 32 controls the motor 9 via control line 31 in such a way that the motor 9, via the gear mechanism 11, moves a dosing piston 13 in the direction R so that a desired dose of medicament is ejected from the medicament container via the connection line 22 into the mouthpiece 24.

The motor 9 is also connected via the gear mechanism 11 to an indicator piston 15 which is part of an indicator device 16.

The movements of the shaft of the motor 9 are conveyed via the gear mechanism 11 not only to the dosing piston 13 but also to the indicator piston 15, which moves an indicator disk 17 in the direction V. The indicator disk 17 has two areas 17a and 17b. The area 17a is colored green for example, and the area 17b is colored red for example.

Each actuation of the actuating button 8 thus triggers the control device 30 via the sensor line 29, and the control device 30 controls the running of the motor 9. The running of the motor 9 is in turn conveyed via its shaft to the gear mechanism 11, which drives both the dosing piston 13 and the indicator piston 15. Each actuation pushes the indicator disk 17 one step further in the direction V; the current position of the indicator disk 17 thus represents the cumulative total usage value. The patient can read this value off through the viewing window 19. In the present example of using the invention, the current total usage value is one of two possible values which are represented by the colors green and red. The color green in this case signifies that the current total usage value has not yet exceeded a minimum total usage value and that there is a high degree of probability that the function of the medicament dispenser 5 is unimpaired.

The color red signifies that the current value of the total usage value has reached or exceeded a minimum total usage value and that the patient should have his medicament dispenser 5 inspected in order to ensure that it continues to function reliably.

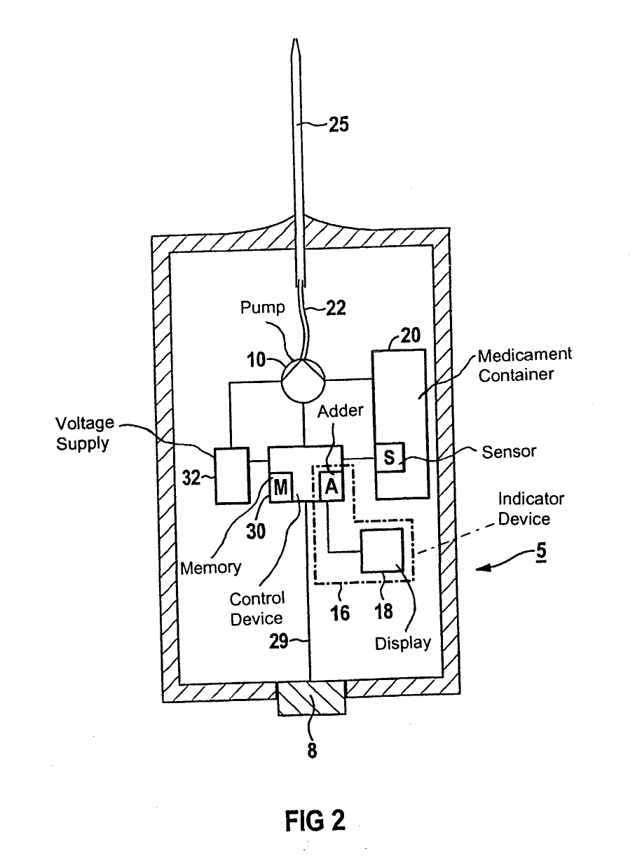

The medicament dispenser 5 in FIG. 2 is designed as an injection device. The medicament container 20 is filled with the medicament which is to be administered. An actuation of the actuating button 8 sends a signal to the control device 30 via the sensor line 29. The control device 30 is connected to the voltage supply 32, to the sensor S of the medicament container, and to the pump 10. The control device 30 controls the pump 10 which, via the connection line 22, delivers the desired amount of medicament, removed from the medicament container 20, to the injection needle 25.

The indicator device 16 includes an adder A which is advantageously a component part of the control device 30, and a display 18 from which the current total usage value can be read off.

In the present illustrative embodiment of the invention, the adder A can form one total usage value or even two total usage values, which can be shown on the display 18.

A first possible usage value is the dose of the medicament which is ejected into the injection needle 25 upon each actuation of the actuating button 8. These individual doses can be recorded in the control device 30 and processed by the adder A to give the total usage value “Total dose”.

Furthermore, the control device 30, using the sensor S, can also total, as a usage value, the amount of medicament removed from the medicament container 20 upon each actuation of the actuating button 8 to give the total usage value “Total amount of medicament removed”. One or both of these total usage values can be shown on the display 18 and read off by the patient.

One or more minimum total usage values are stored in a memory M which is advantageously included in the control device 30, and the control device compares these minimum total usage values with the corresponding current total usage value or values which have been formed by the adder A. If one or more current total usage values exceed(s) the corresponding minimum total usage value or values stored in the memory M, this fact is indicated to the patient on the display 18.

We claim:

1. A medicament dispenser, comprising:
a dosing device; and
an indicator device for forming and displaying a total usage value obtained by totaling at least one usage value assigned to each actuation of said dosing device;
said indicator device designed for indicating when said total usage value exceeds a minimum total usage value; and
the minimum total usage value being derived from an operating period during which there is a high degree of probability that the medicament dispenser operates free from error.

2. The medicament dispenser according to claim 1, wherein the minimum total usage value has been determined in advance by performing a test series.

3. The medicament dispenser according to claim 1, further comprising a medicament container connected to said dosing device and to said indicator device.

4. The medicament dispenser according to claim 1, wherein said dosing device includes at least one motor connected to said indicator device.

5. The medicament dispenser according to claim 4, further comprising a gear mechanism connecting said indicator device to said motor of said dosing device.

6. The medicament dispenser according to claim 1, further comprising:
a medicament container connected to said dosing device and to said indicator device;
said indicator device designed as a counting device forming said total usage value by determining a total amount of medicament removed from said medicament container.

7. The medicament dispenser according to claim 1, wherein said indicator device is designed as a counting device that forms said total usage value by determining a total period of use.

8. The medicament dispenser according to claim 1, wherein said indicator device includes an indicator disk for displaying a current value of said total usage value.

9. The medicament dispenser according to claim 8, wherein said indicator disk is divided into at least two areas each assigned to a value of said total usage value.

10. The medicament dispenser according to claim 8, wherein said indicator disk is divided into at least two areas each assigned to a value range of said total usage value.
